(19) United States
(12) Patent Application Publication
(10) Pub. No.: US 2015/0057738 A1
(43) Pub. Date: Feb. 26, 2015

(54) CATHETER SYSTEM WITH MOVABLE SLEEVE

(71) Applicant: BIOTRONIK AG, Buelach (CH)

(72) Inventors: Markus Hepke, Zuerich (CH); Matthias Wesselmann, Ruedlingen (CH); Fabian Eckermann, Weisslingen (CH)

(21) Appl. No.: 14/457,410

(22) Filed: Aug. 12, 2014

Related U.S. Application Data
(60) Provisional application No. 61/867,626, filed on Aug. 20, 2013.

(51) Int. Cl. A61F 2/966; A61F 2/24; A61F 2/82

(52) U.S. Cl. CPC A61F 2/966; A61F 2/82; A61F 2/24; A61F 2/2436; A61F 2/958
USPC 623/1.11

(57) ABSTRACT

A catheter system for inserting a self-expanding stent into a bodily vessel. The catheter (1) has a movable sleeve (30), which holds the self-expanding stent in the space (2) when being positioned in the bodily vessel. As the self-expanding stent is released, the sleeve is pushed back by means of the application of fluid to the balloon (20). Here, the movable sleeve (30) can be moved proximally or distally in a precise manner by adjusting the pressure ratio in the balloons (20, 23). A controlled release and renewed capture of the self-expanding stent are possible accordingly.

### CROSS REFERENCE TO RELATED APPLICATIONS

This invention claims priority to U.S. provisional patent application Ser. No. 61/867,626 filed Aug. 20, 2013; the entire content of which is herein incorporated by reference in its entirety.

### TECHNICAL FIELD

The invention relates to a catheter system suitable for insertion into a bodily vessel, comprising a catheter having a functional component and a movable sleeve in a distal region, wherein a diameter of the sleeve corresponds at least to the diameter of the functional component and a length of the sleeve is greater than the length of the functional component, further wherein the sleeve covers the functional component when the catheter system is in a state for introduction of the catheter into the bodily vessel, further wherein the catheter at least in the distal region has a shaft having a first, inner lumen and an outer, second lumen.

BACKGROUND

Catheters, in particular wire-guided catheters, are used broadly in predominantly medical applications and interventions. Wire-guided catheters are used in particular in angioplasty and in the implantation of stunts. In applications of this type, the catheter is positioned with the aid of a guide wire introduced beforehand into the body of the patient. The distal tip of a catheter guide wire is generally very flexible, such that it can be bent and rotated so to be advanced to the desired point in the body of the patient, for example within a blood vessel. A wide range of wire-guided catheter types are known, for example interventional catheters, balloon catheters, and catheters for the application for self-expanding stents.

Interventional catheters can cover a broad spectrum of what is known as interventional cardiology or radiology. This spans vascular interventions (such as angioplasty of vessels or the fitting of filters in bodily vessels), tumor ablation interventions, in which tumors are destroyed locally by cold temperatures, for example, and interventions controlled by imaging methods, such as a biopsy.

### INTRODUCTION TO THE INVENTION

The present invention will be described hereinafter primarily on the basis of the example of vascular interventions, in particular on the basis of catheter systems for insertion of self-expanding stents, but is not limited hereto. The invention is suitable for all catheter systems having functional components in the distal region, in which the functional components have to be covered by a movable sleeve when the catheter is introduced into the respective bodily vessel to be treated at the treatment site.

Vascular interventions form a large area of interventional radiology. Here, closed vessels are often reopened and expanded. Balloon catheters are often used to expand closed bodily vessels. A balloon catheter of this type has, at its distal end, a balloon that can be filled with fluid. This is guided under radiological control into the bodily vessel to the constricted point. There, the balloon is expanded by being acted on by fluid and thus expands the surrounding bodily vessel.

Within the scope of this application, the proximal end of a catheter is understood to mean the end that lies in the hand of the operator outside the body/the bodily vessel. The distal end of the catheter is accordingly the tip of the catheter guided into the bodily vessel. The position-related references “proximally” (closer to the operator) and “distally” (closer to the tip of the catheter located in the bodily vessel) are to be understood accordingly.

Within the scope of this application, the terms expansion/expand, dilation/dilate and inflation/inflate with regard to the balloon or a stent are to be considered as synonyms, wherein the term expansion/expand will be used within the scope of this application.

In order to avoid a renewed constriction of expanded vessels, a stent is often inserted into the bodily vessel at the affected point, A stent is generally a small lattice-like structure in a form similar to a tube, made of metal or plastic. A stent of this type can be introduced into the bodily vessel either with a balloon catheter or with a catheter for the application of self-expanding stents.

With the use of a balloon catheter, the stent is placed over the balloon (the term “crimped” often being used in the prior art), where it is secured in a suitable manner. Due to the expansion of the balloon at the point of the constriction, the stent is expanded and, once the pressure has been released and the balloon has been refolded accordingly, remains in the bodily vessel close to provide support. Here, the expansion of the vessel and the placement of the stent can be carried out in a single step or in successive steps.

Self-expanding stents consist of a shape-memory material, in particular special metal alloys, such as the nickel/titanium alloy known as nitinol. The self-expanding stent, at body temperature, has the desired form necessary to support the bodily vessel. At lower temperature, it is placed over a catheter for the application of self-expanding stents, is compressed, and is held there in a compressed form by a suitable means. At the site of the application in the bodily vessel, these means are removed and the self-expanding stent adopts its original form and supports the vessel.

Self-expanding stents are also known for the implantation of artificial heart valves. Here, the artificial heart valves are sewn directly to the self-expanding stent. The self-expanding stent is then placed in the body together with the heart valve in a manner similar to that described above.

The present invention describes a catheter system comprising a catheter having a functional component in the distal region, and is not limited to self-expanding stents for the treatment of constrictions of a bodily vessel or self-expanding stents having artificial heart valves, but is suitable for these applications in particular.

With the application of a self-expanding stent, the guide wire, which is equipped with X-ray markers or is completely visible under X-ray, is already located in the bodily vessel. The catheter is introduced via this guide wire with the tip to the front. Here, the tip of the catheter is very flexible so to avoid injuring the walls of the bodily vessel and is coated in such a way that it can slide in the bodily vessel without friction where possible. In addition, it is visible under X-ray. The guide wire is guided in the first, inner lumen of the catheter.

Within the scope of this application, the lumen for the guide wire is always referred to as the inner lumen (and accordingly inner shaft), independently of the coaxial or parallel arrangement of further lumens.

The catheter is placed with the aid of the guide wire such that the stent is in the desired position in the vessel, In order to ensure reliable placement, the shaft or the movable sleeve has appropriate X-ray markers. The self-expanding stent is fixed on the catheter by the movable sleeve. If the sleeve is moved away accordingly, the self-expanding stent adopts its original form and is released or implanted in the bodily vessel.

Various solutions are known in the prior art for release of a self-expanding stent. In the simplest case, the movable sleeve over the stent is formed as a tube, which surrounds the catheter externally. To release the stent, this outer tube is simply retracted. This sleeve is common in particular with what are known as over-the-wire catheters. In an over-the-wire catheter system, the inner lumen for the guide wire is arranged coaxially. The guide wire is located in the inner shaft with the inner lumen, and the self-expanding stent is located on said guide wire at the distal end and is surrounded by the outer tube as a movable sleeve.

EP 2 329 799 discloses a similar catheter system, but for what is known as a monorail catheter, also referred to a rapid-exchange catheter. In catheter systems of this type, the guide wire, in contrast to over-the-wire catheters, does not run along the entire length in the coaxial inner guide wire lumen of the catheter, but is guided out from the guide wire lumen close to the distal end of the catheter. For this purpose, the catheter has a guide wire exit at the corresponding point. In order to ensure the retraction of the outer tube in a system of this type, the catheter system described in EP 2 329 799 has a cutting device for the outer tube.

In the two described prior art embodiments, the movement of the sleeve is conveyed by a mechanical transmission of force. The outer tube is pulled mechanically from the self-expanding stent, the retaining force on the catheter thus disappears, and the stem is released in the bodily vessel.

Another system for releasing a self-expanding stent is described in WO 2006/089517. Here, the force for releasing the stent is transmitted hydraulically. Here, either the self-expanding stem is slid out of the sleeve surrounding it by means of a piston and hydraulic transmission of force, or the sleeve surrounding the stent is pressed back proximally by means of a piston and hydraulic transmission of force. The movable sleeve here has a greater length than the stent and is terminated at its proximal end with an annular seal, which seals with respect to the inner shaft. An annular seal is likewise located at the proximal end of the stent, such that a space is formed between the two annular seals and the movable sleeve and can be acted on by a fluid via the inner lumen of the inner shaft. When this space is acted on by a fluid, the movable sleeve is thus pressed distally in the manner of a piston and is moved away over the stent, which remains in position. The self-expanding stent is in this way released in the bodily vessel.

Both catheter systems known in the prior art for releasing a self-expanding stem have disadvantages. The mechanical release of the self-expanding stent is accompanied by high frictional forces. This is particularly true when the stent is to be released in a tightly winding bodily vessel. These high frictional forces and the application of force necessary on the part of the operator hinder the accurately positioned release of the stent. The catheter system disclosed in WO 2006/089517 with the hydraulic release of the stent is difficult to manufacture due to the necessary ring seals. Furthermore, the ring seals, as friction seals, have to be adapted carefully and have to be checked for tightness. In addition, this design is relatively large.

The object of the present invention is to provide an alternative catheter system of the type described in the introduction. The described disadvantages of the prior art are to be avoided here to the greatest possible extent.

### SUMMARY

The stated object is achieved by a catheter system suitable for insertion into a bodily vessel, including a catheter having a functional component and a movable sleeve in a distal region, wherein a diameter of the sleeve corresponds at least to the diameter of the functional component and a length of the sleeve is greater than the length of the functional component, further wherein the sleeve covers the functional component when the catheter system is in a state for introduction of the catheter into the bodily vessel, further wherein the catheter at least in the distal region has a shaft having a first, inner lumen and an outer, second lumen, characterized in that the outer second lumen is fluidly connected to at least one first balloon, and the first balloon is connected to the sleeve in such a way that a change to a fluid-filled volume of the first balloon induces a movement of the sleeve. Further advantageous embodiments of the invention are discussed herein.

In accordance with the invention, the outer, second lumen is fluidically connected to at least one first balloon, and the first balloon is connected to the sleeve in such a way that a change to the fluid-filled volume of the first balloon induces a movement of the sleeve. The basic concept of the invention consists in using one or more balloons to move the sleeve and therefore to expose the functional component.

The use and the production of balloon catheters are established in the prior art. According to the present invention however, a stent is not expanded and pressed into the vessel wail by means of expansion of a balloon. In accordance with the present invention, the sleeve is moved away from the functional component via the expansion of one or more balloons, such that said component can perform its function in the bodily vessel. The force for releasing the self-expanding stent is thus transmitted hydraulically via the expansion of at least one balloon.

The present invention therefore has various advantages. On the one hand, the force for releasing the functional component is transmitted hydraulically. The balloon or balloons can be easily acted on by fluid in a manner controlled by the operator and can thus move the sleeve away from the functional component. The functional component is thus released without a great application of force by the operator, whereby the precision of the release process is considerably improved. On the other hand, the present invention uses a simple and established technique for hydraulic release of the functional component. Complicated seals and a bulky and inflexible design as in the prior art are unnecessary as a result of the present invention.

Within the scope of the invention, a functional component is understood to mean an element in the distal region of the catheter that is to perform a specific function in the bodily vessel. A functional component of this type may be an implant, for example, which is inserted and implanted in the bodily vessel via a catheter system. The implant, such as a stent, then performs its function in the bodily vessel, for example its supporting function. Within the meaning of the invention, besides stents, in particular self-expanding stents, expandable balloons, imaging instruments such as ultrasound probes or miniature cameras, or other instruments such as filters can also be considered as functional components. Within the meaning of the invention, a functional component is always located however in the distal region of the catheter and is covered by a movable sleeve when introduced and placed in the bodily vessel. Here, the functional component is not necessarily part of the catheter, but can also be arranged thereon or attached thereto.

Within the scope of this invention, a movable sleeve is understood to mean any type of tubular component that is suitable for surrounding the self-expanding stent. In the simplest case, the movable sleeve is a tube piece, which is at least as long as the self-expanding stent. Here, the movable sleeve can be formed from continuous material or can also be provided with recesses (for example slits). A tubular lattice is likewise possible as a movable sleeve if sufficient rigidity is provided.

In accordance with a preferred embodiment of the invention, the first balloon is connected to the part of the sleeve that projects beyond the length of the functional component when the catheter system is in the state for introduction of the catheter into the bodily vessel.

The movable sleeve is longer than the respective functional component and advantageously covers the component completely when the catheter is introduced into the bodily vessel. The sleeve ensures that the functional component remains in its position. Furthermore, the movable sleeve ensures that, as the catheter is introduced into the bodily vessel, the functional component is not damaged or functionally impaired, and particularly that the bodily vessel is not injured as a result of the insertion of the functional component. The part of the movable sleeve projecting beyond the functional component is connected in this embodiment to the balloon in a suitable manner. If, here, the balloon lumen (the interior of the first balloon or the first balloons) is acted on by fluid and is therefore expanded, a movement of the sleeve will be induced in such a way that the functional component is covered by the sleeve to a reduced extent. The functional component is thus exposed.

The balloon is advantageously adhesively bonded or welded to the part of the sleeve that projects beyond the length of the functional component when the catheter system is in the state for introduction of the catheter into the bodily vessel. Here, commercially available instant adhesives and other reactive glues, which preferably cure via a chemical reaction, are suitable as adhesives, for example. This curing process can be initiated by means of UV irradiation or surface and environment contact, or can be based on a 2-component system that can be cured in a manner accelerated by heat. These adhesives include, for example, acrylates, cyanoacrylates, epoxy adhesives or polyurethanes.

The sleeve is preferably movable along the catheter axis.

Within the scope of the invention, the catheter axis is understood to mean the central longitudinal axis of the catheter. In the case of an over-the-wire catheter with a coaxial arrangement of the first, inner lumen with the guide wire, and with an inner shaft with surrounding second, outer lumen for exposing the balloon to the action of a fluid, and with an outer shaft, the catheter axis is the longitudinal axis of the first, inner lumen. The guide wire runs along the catheter axis in a catheter of this type.

In this embodiment of the invention, the sleeve is movable along the catheter axis and, as the stent is released, is displaced along the catheter axis proximally or distally accordingly by means of the application of fluid to the first balloon. The movement along the catheter axis is also a movement along the bodily vessel as the stent is released. It is accordingly ensured in this embodiment that the bodily vessel cannot sustain any damage as a result of the movement of the sleeve as the stent is released.

The functional component is preferably a stent, in particular a self-expanding gent, or an expandable third balloon, in particular an expandable third balloon with a coating.

Within the scope of the invention, a self-expanding stent is understood to mean any type of implants formed from a shape-memory material, in particular a metal shape-memory alloy (for example nitinol). In particular, self-expanding stents are to be understood as are used for the treatment of a stenosis in a bodily vessel. The present invention is also particularly suitable for self-expanding stents with artificial heart valves.

Within the scope of the invention, an expandable balloon is understood to n mean any type of balloon that is expanded as a result of being acted on by a fluid.

In this preferred embodiment of the invention, the advantages of the invention are particularly relevant. The movable sleeve advantageously covers the self-expanding stent completely when the catheter is introduced into the bodily vessel. The sleeve ensures that the stent remains pressed onto the shaft, that is to say “crimped” onto the shaft. It is thus ensured that the diameter of the catheter system is small enough for the catheter system to be introduced and displaced even in small, winding bodily vessels. Due to the movement of the sleeve, the self-expanding stent is covered by the sleeve to a reduced extent, whereby the retaining force on the stent disappears and the stent is expanded and thus released.

Similar advantages are provided if the functional component is a stent or expandable balloon and if these are provided with a coating containing an active ingredient. The movable sleeve ensures that the coating containing active ingredient is not damaged as the catheter is introduced and protects the layer containing active ingredients. Once the catheter has been positioned, the sleeve is moved in such a way that the coated stent or coated balloon is no longer covered by the sleeve, and the active ingredient is released directly at the desired site.

In a preferred embodiment of the invention, the functional component is a self-expanding stent. The self-expanding stent is particularly preferably positioned on the inner shaft or connected thereto by means of a proximal stop. In this embodiment of the invention, it is ensured that the stem remains in its position in the catheter, even as the catheter is introduced and advanced into the bodily vessel. Here, the stop may advantageously be made of material that is visible under X-ray, such that the operator can optimally check the position of the stent at all times during insertion and advance of the catheter and in particular during release of the stent. This embodiment of the invention is in particular advantageous in combination with a sleeve that is movable in the proximal direction. As a result of the proximal movement of the sleeve during release, a force acts in the proximal direction on the stent as a result of the friction. This proximal movement of the stent is hindered by the stop however. A distal force in this embodiment of the invention does not act on the stent. This embodiment of the invention thus ensures the maximum accuracy when releasing and placing the stent in the bodily vessel. In this embodiment of the invention, the sleeve is withdrawn proximally over the stent as a result of the expansion of the balloon.

In a particularly preferred embodiment of the invention, the sleeve is connected to at least one second balloon, preferably via an intermediate shaft, wherein the catheter has a third, outer lumen, at least in the distal region, wherein the third, outer lumen is fluidically connected to the second balloon.

Within the scope of this application, an intermediate shaft is understood to mean any device-side connection between the movable sleeve and the second balloon suitable for ensuring a sufficient transmission of force from the second balloon to the movable sleeve and for conveying a movement of the sleeve as a result of the change to the second balloon lumen. Here, the second balloon lumen is similarly understood to mean the interior of the second balloon or balloons. In the simplest case, the intermediate shaft consists of the same material as the inner and/or outer shaft, but preferably has a slightly greater wall thickness and/or strength and/or buckling strength.

Here, the first balloon is preferably distanced from the second balloon in the direction of the catheter axis.

This embodiment of the invention is particularly suitable for the implantation of self-expanding stents having an artificial heart valve. This embodiment with a first and a second balloon or a first group and a second group of balloons, which in particular are distanced from one another along the catheter axis, allows the controlled movement of the sleeve both in the distal and in the proximal direction. The position of the sleeve can be controlled precisely here by the operator in a simple manner and without mechanical application of force.

The first and the second balloon are arranged expediently here such that an application of fluid to one balloon combined with a venting of the other balloon induces a movement of the sleeve in the distal direction, and the application of fluid to the second balloon in conjunction with a venting of the first balloon induces a movement of the sleeve in the proximal direction. The movement and position of the sleeve can be controlled in this way by the first and second balloon by means of the application of fluid and the venting process. If both the first and second balloon are acted on by fluid with the same pressure, the position of the movable sleeve does not change. If the pressure or the volume in the distal balloon increases and in the proximal balloon reduces, a movement of the sleeve in the proximal direction is accordingly induced. Similarly, an increase of pressure or volume in the proximal balloon and a reduction of pressure or volume in the distal balloon cause a movement in the distal direction.

The second balloon(s) is/are connected here either directly to the movable sleeve or is/are connected to the movable sleeve via the intermediate shaft.

As already mentioned, in this embodiment of the invention, the movable sleeve can be moved hydraulically both proximally and distally. In one direction, it is moved away from the self-expanding stent accordingly and releases it accordingly. The self-expanding stent can be captured again by the movement of the sleeve in the opposite direction.

The movable sleeve is preferably produced from solid polymers that are not sensitive to moisture, such as polyamide (10-)12, trogamide, cristamide or aramide, polyesters such as polyethylene terephthalate (PET) and/or polyimide. In this case, it may also be expedient to mix together different materials in order to achieve a suitable mixture having high strength and good sliding properties. The coextrusion of the aforementioned materials or the reinforcement by means of metal wires (for example coils and braids) is also expedient in order to influence specific properties, such as surface hardness and friction at the inner faces, and to provide sufficient overall strength and flexibility.

In particular if the functional component is a self-expanding stent, it may be expedient to design the movable sleeve rather as a lattice structure.

The intermediate shaft is preferably formed from a material similar to the rest of the catheter shafts, but preferably has a slightly higher strength and/or buckling strength. The catheter is expediently made of polyethylene (PE), vinyl polymer, polyesters such as PET, polyamide such as polyether block amides (PEBA) and/or polyimide or polyether ether ketone (PEEK). Here too, expedient suitable materials can be coextruded or reinforced in order to obtain good sliding properties with sufficient rigidity.

The self-expanding stent is particularly preferably suitable for the treatment of a stenosis in a bodily vessel. The self-expanding stent advantageously carries an artificial heart valve and/or is suitable for use in the physiological position of one of the heart valves.

The advantages of the present invention are particularly relevant when the self-expanding stent has an artificial heart valve and/or is used in the physiological position of a heart valve.

In the case of heart valves, such as the aortic valve, malfunctions may occur as a result of deposits (calcifications). In this case, the valve no longer closes or opens completely. In such a case, an artificial heart valve can be used. To this end, a self-expanding stent usually placed in the physiological position of the natural heart valve. This stent carries artificial heart valve, which takes over the function of the natural valve.

With a method of this type, the exact positioning of the stent, in particular of the stent with the artificial heart valve, is key. Here, the advantages of the present invention are particularly relevant. Due to the hydraulic transmission of force as the sleeve is moved by means of expansion of a balloon, the release of the self-expanding stent can be controlled very accurately and precisely. In particular, the embodiment of the invention with at least one first balloon and at least one second balloon allows very effective control of the positioning by the operator. By means of suitable X-ray markers, for example on the proximal stop, the position of the self-expanding stent can be monitored permanently during the release process. Due to the fact that the sleeve is pushed back by the second balloon, the possibility of recapturing the partly released stent if positioned incorrectly and of correcting the positioning is additionally provided.

In another preferred embodiment of the invention, the expandable third balloon has a coating that contains a therapeutically effective substance, in particular a therapeutically effective substance for treating a stenosis in a bodily vessel. In this embodiment of the invention, the functional component is a balloon coated with active ingredient. The active ingredient is only to be released at the treatment site with expansion of the balloon. The movable sleeve accordingly prevents damage to the coating as the catheter is introduced into the bodily vessel and avoids damage to the patient as a result of incorrectly released active ingredient. In this embodiment also, the movable sleeve is moved with respect to the expandable third balloon as a result of the application of a fluid to the first balloon and/or second balloon. The operator can move the sleeve, similarly to the application of the active ingredient, by means of fluid application and pressure changes caused thereby.

The present invention in particular has the advantage of accurate positioning of a self-expanding stent. Furthermore, simple handling of the catheter system with exposure of the functional component is ensured by means of the hydraulic transmission of force. In addition, the catheter system according to the invention is characterized by a simple and tested manufacture by the use of expandable balloon for moving the sleeve. Due to the avoidance of friction seals and pistons, as are provided in a catheter system according to the prior art, the catheter system according to the invention can be smaller and less rigid. The catheter system according to the invention can accordingly be used even in small and winding bodily vessels.

The present invention will be explained in greater detail hereinafter on the basis of the exemplary embodiment illustrated in the figure, in which the functional component is a self-expanding stent.

### DESCRIPTION OF THE DRAWINGS

In the drawings:

FIG. 1 shows an exemplary embodiment of a catheter system according to the invention in the state for introduction of the catheter into the bodily vessel,

FIG. 2 shows the exemplary embodiment according to FIG. 1 with partly released stent, and

FIG. 3 shows the exemplary embodiment according to FIG. 1 with released stent.

### DETAILED DESCRIPTION

Here, only the distal part of the exemplary embodiment is illustrated in the figures.

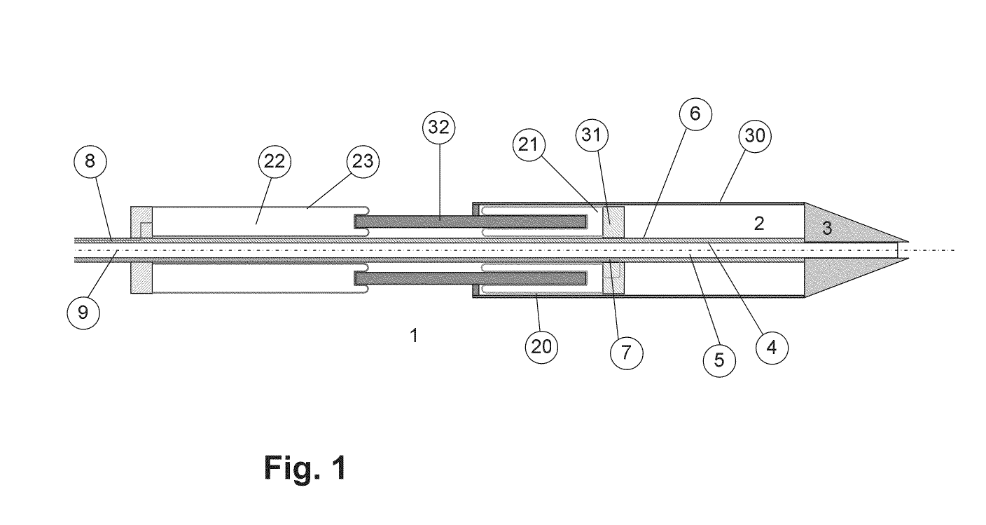

FIG. 1 shows an exemplary embodiment of a catheter system according to the invention in the state for introduction of the catheter 1 into the bodily vessel. The self-expanding stent, for example a self-expanding stent having an artificial heart valve, is located in the cavity 2 and is covered completely by the sleeve 30. The sleeve 30 prevents the stent from expanding. The stent remains in the compressed state and thus has a slightly smaller diameter than the catheter 1 itself and can thus be inserted with the catheter 1 into the bodily vessel.

The catheter 1 has an inner shaft 4, which surrounds the first, inner lumen 5. The inner lumen 5 is generally used to receive the guide wire. The catheter shaft 6 itself is formed as a shaft having three different lumens, the illustrated inner lumen 5 for the guide wire and the outer, second lumen 7 and the outer third lumen 8 (neither of which is illustrated in detail). Both lumens 7 and 8 can be arranged coaxially with or parallel to the catheter axis 9.

The second, outer lumen 7 is fluidly connected to the interior 21 of the first balloon 20, whereby the first balloon 20 can be acted on by fluid.

The intermediate shaft 32 is used to move the sleeve 30 and is connected at its proximal end to a second balloon 23. The interior 22 of the second balloon 23 is fluidly connected via the third, outer lumen 8 to a second fluid source and can be acted on by fluid separately from the first balloon 20.

The self-expanding stent (not illustrated) is located in the space 2, wherein the position of the stent when the catheter is introduced into bodily vessel is exactly defined by the movable sleeve 30 over the stent and the proximal stop 31. In the state for introduction of the catheter into the bodily vessel, the sleeve 30 extends over the entire region of the catheter from the stop 31 to the tip 3 of the catheter.

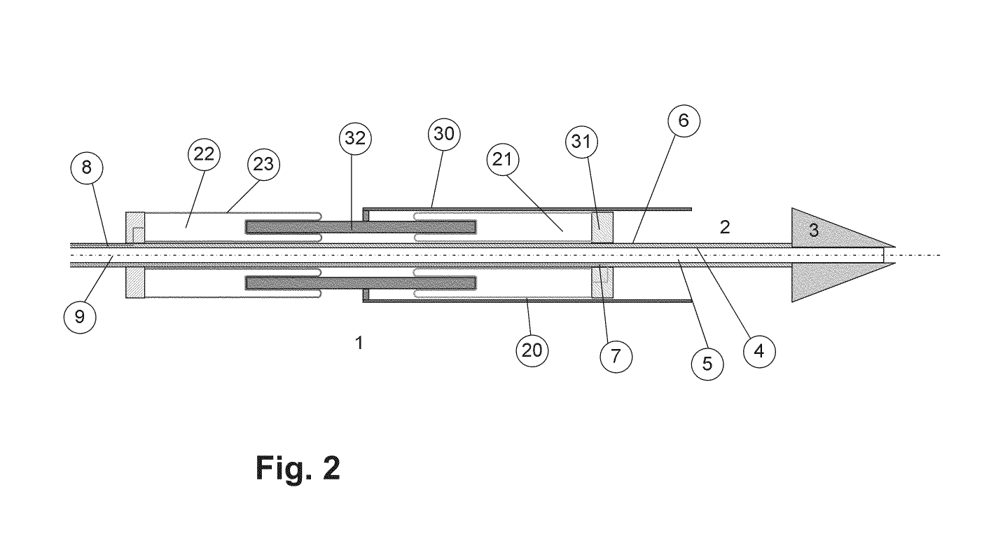

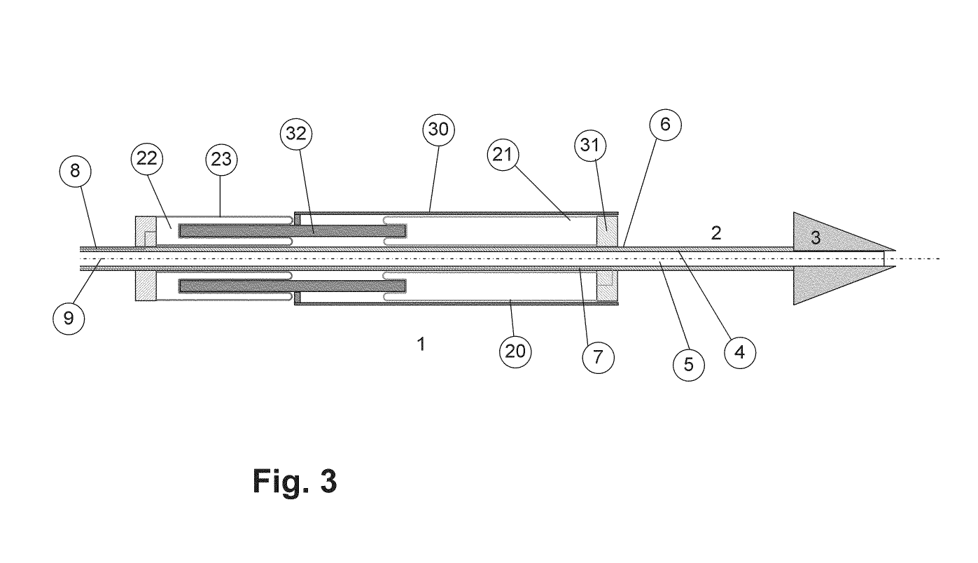

The release of the self-expanding stent in the bodily vessel will be described hereinafter with reference to FIGS. 2 and 3. FIGS. 2 and 3 show the same embodiment of the catheter system according to the invention as shown in FIG. 1. Like parts have been provided with like reference numerals.

As the catheter 1 is introduced into the bodily vessel, the second balloon 23 is acted on by a considerably higher pressure than the first balloon 20. The movable sleeve 30 is thus pushed via the intermediate shaft 32 toward the distal end of the catheter. The movable sleeve 30 thus covers the self-expanding stent completely and holds it in its compressed form on the catheter. By means of the counter pressure in the second balloon 23, the sleeve is prevented from being pulled back as a result of the frictional forces between the catheter and the walls of the bodily vessel or the hemostatic valve of the introducer sheath during introduction of the catheter. The catheter system according to the invention can thus be introduced reliably into the bodily vessel. In the normal case, the pressure in the interior 21 of the first balloon 20 is approximately 0 as the catheter system is introduced into the bodily vessel, that is to say the interior 21 of the first balloon 20 is hardily filled with fluid.

When the catheter system is located in the bodily vessel, the position of the self-expanding stent can be checked continuously by means of the tip 3, which is visible under X-ray, by means of X-ray markers in the movable sleeve 30 and/or in the stop 31 and/or in the tip 3 and/or on/in the inner shaft 4. If the self-expanding stent is located in the desired position, the stent can be released. To this end, the interior 21 of the first balloon 20 is acted on by fluid, whereas the pressure in the interior 22 of the second balloon 23 is reduced at the same time. Both balloons 20, 23 are connected via the intermediate shaft 32 to the movable sleeve 30. The rise in pressure in the interior 21 of the first balloon 20 leads to an enlargement of the volume. Due to the enlarged volume 21 of the first balloon 20, the movable sleeve 30 moves proximally via the intermediate shaft 32.

The counter pressure and the volume of the second balloon 23 are reduced during this process. Due to the simultaneous build-up of pressure in the first balloon 20 and breakdown of pressure in the second balloon 23, a very controlled movement of the sleeve 30 is provided. The self-expanding stent can accordingly be released from the space 2 in a very controlled manner.

FIG. 2 shows a corresponding intermediate state, where, in comparison to FIG. 1, the pressure in the interior 21 of the first balloon 20 has risen and the pressure in the interior 22 of the second balloon 23 has reduced. In the state shown in FIG. 2 the pressure in the interior 21 of the first balloon 20 and the pressure in the interior 22 of the second balloon 23 are identical. The sleeve 30 has accordingly moved proximally in a controlled manner and has released half of the self-expanding stent.

FIG. 3 shows the state of the complete release of the self-expanding stent. The pressure conditions in the interior 21, 22 of the two balloons 20, 23 are inverted compared to the state in FIG. 1. The second balloon 23 is pressureless, whereas the first balloon 20 is acted on by fluid and is fully expanded. Accordingly, the intermediate shaft 32 and movable sleeve 30 are moved back proximally to the maximum extent.

Here, the catheter system according to the invention also offers the possibility of renewed capture and repositioning of the self-expanding stent. In this case, only the pressure conditions in the interior 21, 22 of the balloons, 20, 23 have to be reversed again. The pressure in the interior 22 of the second balloon 23 is increased again by means of the application of fluid, whereas the pressure in the interior 21 of the first balloon 20 is reduced again. The state of the catheter system from FIG. 3 transitions similarly via the state from FIG. 2 back into the state of the catheter system as illustrated in FIG. 1. Here, the stent can only be captured again provided it is held again on the catheter (for example is located again to an extent of one third beneath the sleeve 30).

Due to the control of the two pressures or volumes in the interior 21, 22 or of the pressure conditions of the balloons 20, 23, any position of the sleeve can be set precisely. The force h which the sleeve 30 is moved, for example when being pushed back over the already expanded stent, can be determined by the cross-sectional area of the intermediate shaft 32. The force is given directly from the product of the pressure difference and the cross-sectional area of the intermediate shaft 32.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A catheter system suitable for insertion into a bodily vessel, comprising a catheter having a functional component and a movable sleeve in a distal region, wherein a diameter of the sleeve corresponds at least to the diameter of the functional component and a length of the sleeve is greater than the length of the functional component, further wherein the sleeve covers the functional component when the catheter system is in a state for introduction of the catheter into the bodily vessel, further wherein the catheter at least in the distal region has a shaft having a first, inner lumen and an outer, second lumen, characterized in that the outer second lumen is fluidly connected to at least one first balloon, and the first balloon is connected to the sleeve in such a way that a change to a fluid-filled volume of the first balloon induces a movement of the sleeve.

2. The catheter system of claim 1, characterized in that the first balloon is connected to a part of the sleeve that projects beyond the length of the functional component when the catheter system is in the state for introduction of the catheter into the bodily vessel.

3. The catheter system of claim 1, characterized in the sleeve is movable along the catheter axis.

4. The catheter system of claim 1, characterized in that the functional component is a self-expanding stent or an expandable third balloon with a coating.

5. The catheter system of claim 4, characterized in that the self-expanding stent is positioned on the shaft and/or is connected to the shaft by means of a proximal stop.

6. The catheter system of claim 4, characterized in that e self-expanding stent is suitable for treating a stenosis in a bodily vessel.

7. The catheter system of claim 4, characterized in that the self-expanding stent carries an artificial heart valve and/or is suitable for use in the physiological position of one of the heart valves.

8. The catheter system of claim 4, characterized in that the expandable third balloon has a coating, which contains a therapeutic effective substance, in particular a therapeutically effective substance for treating a stenosis in a bodily vessel.

9. The catheter system of claim 1, characterized in that the sleeve is connected to at least one second balloon, optionally via an intermediate shaft, wherein the catheter has a third, outer lumen at least in the distal region, wherein the third, outer lumen is fluidly connected to the second balloon.

10. The catheter system of claim 1, characterized in that the first balloon is distanced from the second balloon in the direction of the catheter axis.
